(19) United States
(12) Patent Application Publication
(10) Pub. No.: US 2018/0344967 A1
(43) Pub. Date: Dec. 6, 2018

(54) ANESTHETIC DELIVERY SYSTEM

(71) Applicants: Joseph Fisher, Thornhill (CA); James Duffin, Toronto (CA); Joel Ironstone, Toronto (CA)

(72) Inventors: Joseph Fisher, Thornhill (CA); James Duffin, Toronto (CA); Joel Ironstone, Toronto (CA)

(21) Appl. No.: 15/908,089

(22) Filed: Feb. 28, 2018

Related U.S. Application Data
(63) Continuation of application No. 14/389,329, filed on Sep. 29, 2014, now abandoned.
(60) Provisional application No. 61/617,578, filed on Mar. 29, 2012.

(51) Int. Cl. A61M 16/18; A61M 16/00; A61M 16/08; A61B 5/087; A61M 16/01; A61M 16/22; A61B 5/00; A61M 16/10

(52) U.S. Cl. CPC A61M 16/183; A61M 2016/0027; A61M 16/0081; A61M 16/0891; A61B 5/087; A61M 16/0051; A61M 16/0057; A61M 16/01; A61M 16/0003; A61M 16/22; A61M 2016/0039; A61M 2230/437; A61M 16/0833; A61M 16/0093; A61B 5/4821; A61M 16/0075; A61M 16/0066; A61M 2016/102; A61M 2016/1035; A61M 2202/048; A61M 2205/3303; A61M 2205/3334; A61M 2205/3389; A61M 2205/502; A61M 2230/42; A61M 16/18

(57) ABSTRACT

An anesthetic delivery system for use in conjunction with an anesthetic return system for reutilizing anesthetic exhaled by a subject, the system including a measurement system operatively connected to a breathing circuit for continuously measuring at least one flow parameter and anesthetic content of a gas stream reaching the subject and a control system for receiving input from the measurement system and controlling the amount of anesthetic entering the system, the control system including an input device for inputting a setting that corresponds to a desired amount of anesthetic in the gas stream reaching the subject, and utilizing a control algorithm for controlling the amount of anesthetic entering the system based on said desired amount of anesthetic, and flow and anesthetic content parameters as determined by the measurement system, such that the control algorithm is adapted to supplement anesthetic already in gas stream flowing to the subject to attain a level of anesthetic reaching the subject that correspond to the desired amount set via the input device.

### BACKGROUND

### 1. Technical Field

Embodiments of the present invention relate generally to breathing systems and, more particularly, but not exclusively, to a system and method for delivering anesthetic to a medical patient.

### 2. Description of Related Art

Anesthetic delivery systems are used in many surgical procedures to keep the patient being operated upon in an unaware and unconscious state so that the surgery can proceed safely and without interruption. Such systems are also used in non-surgical environments, such as the intensive care unit (ICU) of a hospital or on a battle field during war, where it is desired to keep a patient sedated to assist with comfort, recovery or treatment.

These systems generally contain a closed network of tubes or hoses that connect the patient's airway to a ventilator, which is typically a machine that maintains a flow of air into and out of the lungs of the breathing patient. The ventilator generally contains a compressible chamber or bag to hold a given volume of gas, and a blower or other active device to compress it to push the gas out to the patient. Typically a ventilator also has an input port that brings fresh air into the ventilator to which oxygen and anesthetic gas supplied by a vaporizer may be added. Accordingly, the gas in the ventilator bag generally comprises of a mixture of fresh air, anesthetic gas, and in re-breathing or circle systems, air previously exhaled by the patient.

When the patient exhales, the exhaled (or “expired”) air passes through a tube to the ventilator. When the patient subsequently inhales, the inhaled (or “inspired”) air passes through a tube from the ventilator to the patient. In this way anesthetic gas enters the patient's bloodstream and produces the desired anesthetic effect. Such systems, where exhaled air is received by the ventilator and recirculated, are known as rebreathing systems. Usually a carbon dioxide (CO₂) scrubber is included in the air conduit to remove CO₂ from the system.

A key parameter in this process is “FmA”, which is the percentage concentration by volume of anesthetic in the air inspired by the patient. In a surgical environment, the FmA is monitored and adjusted as needed throughout the surgical procedure by the attending medical staff such as an anesthetist. This figure is generally kept within a specified range, as too low a concentration may result in the patient waking up prematurely, and too high a concentration may lead to dangerous medical complications.

It is often the case during surgery that the anesthetist will discover that the level of anesthesia is too low, risking that the patient will wake up prematurely. As this is a dangerous situation, it is very important that the level of anesthesia, or FmA, be raised to a desired higher level as quickly as possible. However, many anesthetic breathing systems in current use are unable to increase FmA quickly enough to meet the demands of the medical situation. This is illustrated in FIG. 1, which shows a chart of FmA plotted with respect to time, when FmA is raised from an initial low value V₁ to a desired higher value V₂. As illustrated, FmA does not rise instantly or immediately, but rather rises to V₂ only exponentially, over a period of time “dt”. In some situations dt may be as long as several minutes, which is considered unacceptably long as it increases the likelihood that the patient will awaken and create serious medical complications.

This relatively slow rate of increase results from the fact that in order to increase FmA, more anesthetic needs to be introduced into the breathing circuit. Typically, this is accomplished by increasing the speed or flow rate of the ventilator. The ventilator will therefore pump more frequently in a given time period, with each pump delivering more fresh air and accompanying anesthetic.

Further, in the case of closed or partially closed systems, there is a limit on the total volume of gas that can be contained in the system. Any excess gas beyond this limit will get flushed out automatically by an expiratory valve (often in the form of a mushroom or “poppet” valve) that is usually installed for this purpose in one of the gas conduits in the system. As a result, increasing the rate of fresh gas flow and the anesthetic content of fresh gas will cause a lot of the added anesthetic simply to pass through and out of the system at the same rate as it entered, at great waste and cost.

It is noteworthy that it is the volume in a patient's lungs that needs to be raised in concentration which includes not just the amount inhaled in a given breath (the “tidal volume”), but also a further volume of residual air that stays in the lungs and does not get expelled upon exhalation (called the “functional residual capacity”, or “FRC”). The effect of this is that the anesthetic delivered by the ventilator in the title volume has to be at a higher concentration than the desired target (i.e. “V₂”), in order to raise the concentration of the FRC.

It may also be noted that excess anesthetic that is ejected from the system enters the immediate environment, where it constitutes a health hazard when breathed by the medical staff. For this reason some systems incorporate additional components that act to vacuum away this gas and remove the health risk. In either case the net effect of the breathing system being forced to eject anesthetic gas is problematic, as it results in either a health hazard or an increase in the cost of the system.

In a non-surgical environment such as the ICU, the patient sedation level is typically monitored by medical staff such as a nurse or physician's assistant rather than by an anesthetist. Accordingly, the types of anesthetic delivery systems used are generally simpler and easier to operate.

An example of such a system is shown in Lambert, WO 2006/009498, FIG. 2. In this system the breathing tube leading from the patient's mouth contains a gas monitor or sensor, an in-line vaporizer, and an absorption means. The absorption means, also called a reflector, is a type of filter that captures anesthetic gas exhaled by the patient, and releases it back to the patient upon inhalation. The in-line vaporizer functions to vaporize liquid anesthetic supplied from an external container. Past the reflector the breathing tube splits into an inlet tube to receive inhaled gas and an outlet tube to expel exhaled gas. The in-line vaporizer and reflector may be packaged in a common housing, such as the AnaConDa™ device manufactured by Sedana Medical. Another example of a similar system but which uses a more conventional external vaporizer is shown in Psaros, U.S. Pat. No. 5,237,990, FIG. 1.

In the latter anesthetic delivery systems exhaled air is vented and not re-circulated to the patient, while anesthetic gas is mostly trapped by the reflector and returned to the patient in a subsequent inhalation cycle. In this way the costly anesthetic gas is conserved (to the level of efficiency of the reflector), and the amount of gas undesirably released into the environment is reduced.

One problem with this type of system is that it creates a certain amount of dead space in the breathing tube in front of the patient's mouth and may obstruct access to the patient. Accordingly, there is a need for an improved system for anesthetic delivery.

### BRIEF SUMMARY OF INVENTION

According to one aspect, the invention is directed to an anesthetic delivery system for use in conjunction with a ventilator, and a breathing circuit that organizes gas flow in relation to a subject, the breathing circuit operatively associated with an anesthetic return system for re-utilizing anesthetic exhaled by a subject and including an inspiratory limb for directing a gas stream from the ventilator towards the subject, the system comprising:
an anesthetic delivery device for introducing a variable amount of anesthetic into the breathing circuit, the breathing circuit adapted to receive the anesthetic via an anesthetic inlet port in the breathing circuit;
a measurement system operatively connected to the breathing circuit for determining, preferably continuously, at least one measurement parameter describing flow and anesthetic content of the gas stream; and
a control system for receiving input from the measurement system and controlling the amount of anesthetic entering the inlet port from the anesthetic delivery device, the control system operatively associated with an input device for inputting a setting that corresponds to a desired amount of anesthetic in the gas stream reaching the subject, the control system utilizing a control algorithm for controlling the amount of anesthetic entering the anesthetic inlet port based on said desired amount of anesthetic, and flow and anesthetic content parameters as determined by the measurement system, such that the control algorithm is adapted to supplement anesthetic already in gas stream flowing to the subject to attain a level of anesthetic reaching the subject that correspond to the desired amount set via the input device.

The control system is embodied in a controller, for example in the form of a processor, for example a microcontroller.

The measurement system provides output sufficient to compute the amount of anesthetic required to attain a desired amount of anesthetic set via the input device by evaluating and topping-up anesthetic already in the inspiratory gas flow. Optionally the measurement system continuously measures the rate of flow of the gas stream flowing to the subject and its anesthetic concentration.

Optionally, the anesthetic delivery system delivers anesthetic in a vaporized form through the anesthetic inlet port.
Optionally, the inspiratory limb is operatively connected to a homogenizer for distributing the anesthetic vapor in the gas stream flowing to the subject.

Optionally, the breathing circuit directs anesthetic vapor from the anesthetic delivery device into the homogenizer, the anesthetic inlet port located in the homogenizer.

Optionally, the breathing circuit includes a carbon dioxide scrubber and a separate expiratory limb for receiving gas exhaled by the subject, the expiratory limb and carbon dioxide scrubber fluidly connected to the inspiratory limb such that the gas stream flowing to the subject via the inspiratory limb includes exhaled gas containing anesthetic gas exhaled by the subject.

Optionally, the measurement system comprises an anesthetic analyzer for analyzing a concentration of anesthetic in the gas stream flowing towards the subject including anesthetic returned via the anesthetic return system, and a flow sensor for determining a rate of flow of the gas stream to the subject.

Optionally, the flow sensor and the anesthetic analyzer are operatively connected to the inspiratory limb between the anesthetic inlet port and the ventilator.

Optionally, the input device is adapted for setting the desired amount of anesthetic in terms of a selectable concentration of anesthetic in the gas stream reaching the subject.

Optionally, the control algorithm:
a. outputs a value corresponding to a volume of gas inspired in each of a series of respective breaths [i] using input from the measurement system; and
b. uses the volume of each such respective breath [i] and it's pre-top up anesthetic content to compute the amount of anesthetic set to enter the anesthetic inlet port to attain the desired amount of anesthetic in the gas stream reaching the subject.

Optionally, the control system is programmed to send a control signal to the anesthetic delivery device to signal the anesthetic delivery device to deliver an amount of anesthetic corresponding to difference between the desired amount of anesthetic and the amount of anesthetic already in the gas steam from the anesthetic return system in a respective breath [i], in a subsequent breath [i]+1, any requisite addition of anesthetic to attain or maintain the selected concentration of anesthetic in the gas stream delivered to the anesthetic inlet port, added in increments computed on a breath by breath basis, one breath behind.

Optionally, the control algorithm:
a. outputs a value corresponding to a volume of gas inspired measured for each of a series of consecutive time intervals [t] using input from the measurement system; and
b. uses a respective volume of gas measured for a respective time interval [t] and it's pre-top up anesthetic content to compute the amount of anesthetic required to attain the desired amount of anesthetic in the gas stream flowing to the subject;
   the control system programmed to top-up the anesthetic already in the gas stream within a next ensuing time interval [[t+1] or a plurality of next ensuing time intervals, as required, to attain the desired amount of anesthetic in the gas stream reaching the subject.
   Optionally, the anesthetic delivery device is a vaporizer which comprises:
a reservoir containing liquid anesthetic;
a vaporization chamber, to convert the liquid anesthetic into gaseous anesthetic;
a liquid pump, to transfer liquid anesthetic from the reservoir to the vaporization chamber; and
a gas pump, to transfer the gaseous anesthetic from the vaporization chamber to the anesthetic inlet port.

Optionally, the liquid pump is adapted to transfer anesthetic to the vaporization chamber at an adjustable flow rate controlled by the control system.

Optionally, the breathing circuit is operatively connected to a reflector, and a separate expiratory limb receives gas exhaled by the subject, the reflector operatively connected to the expiratory limb for reversibly trapping (e.g. adsorbing) anesthetic in gas exhaled by a subject and between the inspiratory limb and the ventilator, wherein the ventilator drives inspiratory gas through the reflector into the inspiratory limb such the gas stream flowing to the subject via the inspiratory limb includes anesthetic gas trapped by the reflector.

Optionally, the reflector is positioned in the breathing circuit between the inlet port and the ventilator.

Optionally, the reflector is positioned in the breathing circuit between the measurement system and the ventilator and wherein the control system is programmed to top-up the anesthetic returned to the inspiratory limb from the reflector.

Optionally, the reflector is positioned in the breathing circuit between the homogenizer and the ventilator.

According to another embodiment, the invention is directed to a computer program product adapted to control an anesthetic delivery system for use in conjunction with a ventilator, and a breathing circuit that organizes gas flow in relation to a subject, the breathing circuit operatively associated with an anesthetic return system for re-utilizing anesthetic exhaled by a subject and including an inspiratory limb for directing a gas stream from the ventilator towards the subject, the computer program product comprising:
Program code for introducing a variable amount of anesthetic into the breathing circuit, the breathing circuit adapted to receive the anesthetic via an anesthetic inlet port in the breathing circuit;
Program code for handling output from a measurement system operatively connected to the breathing circuit for continuously measuring at least one flow parameter and anesthetic content of the gas stream; and
Program code defining a control system for receiving input from the measurement system and controlling the amount of anesthetic entering the inlet port from the anesthetic delivery device, the control system including an input device for inputting a setting that corresponds to a desired amount of anesthetic in the gas stream reaching the subject, the program code comprising a control algorithm for controlling the amount of anesthetic entering the anesthetic inlet port based on said desired amount of anesthetic, and flow and anesthetic content parameters as determined by the measurement system, such that the control algorithm is adapted to supplement anesthetic already in gas stream flowing to the subject to attain a level of anesthetic reaching the subject that correspond to the desired amount set via the input device.

According to one aspect, the invention is directed to a processor configured for use in conjunction with a ventilator, and a breathing circuit that organizes gas flow in relation to a subject, the breathing circuit operatively associated with an anesthetic return system for re-utilizing anesthetic exhaled by a subject and including an inspiratory limb for directing a gas stream from the ventilator towards the subject, the processor characterized in that it is configured for:
controlling an anesthetic delivery device such that the anesthetic delivery device introduces a variable amount of anesthetic into the breathing circuit, the breathing circuit adapted to receive the anesthetic via an anesthetic inlet port in the breathing circuit;
receiving output from a measurement system operatively connected to the breathing circuit for continuously measuring at least one flow parameter and anesthetic content of the gas stream; and
implementing a control system operatively that receives output from the measurement system and controls the amount of anesthetic entering the inlet port from the anesthetic delivery device, the control system operatively associated with an input device for inputting a setting that corresponds to a desired amount of anesthetic in the gas stream reaching the subject, the control system utilizing a control algorithm for controlling the amount of anesthetic entering the anesthetic inlet port based on said desired amount of anesthetic, and flow and anesthetic content parameters as determined by the measurement system, such that the control algorithm is adapted to supplement anesthetic already in gas stream flowing to the subject to attain a level of anesthetic reaching the subject that correspond to the desired amount set via the input device.

According to a method for delivering anesthetic to a subject, the method adapted for use in conjunction with a ventilator, and a breathing circuit that organizes gas flow in relation to a subject, the breathing circuit operatively associated with an anesthetic return system for re-utilizing anesthetic exhaled by a subject and including an inspiratory limb for directing a gas stream from the ventilator towards the subject, the method comprising:
continuously measuring at least one flow parameter and anesthetic content of the gas stream; and
controlling an anesthetic delivery device adapted for introducing a variable amount of anesthetic into the breathing circuit, the breathing circuit adapted to receive the anesthetic via an anesthetic inlet port in the breathing circuit including:
a) receiving input from the measurement system; and
b) controlling the amount of anesthetic entering the inlet port from the anesthetic delivery device including:
i) receiving input from an input device for inputting a setting that corresponds to a desired amount of anesthetic in the gas stream reaching the subject; and
ii) utilizing a control algorithm for to control the amount of anesthetic entering the anesthetic inlet port based on said desired amount of anesthetic, and flow and anesthetic content parameters as determined by the measurement system, such that the control algorithm is adapted to supplement anesthetic already in gas stream flowing to the subject to attain a level of anesthetic reaching the subject that correspond to the desired amount set via the input device.

According to another aspect, the invention is directed to an anesthetic delivery system for use in conjunction with a ventilator, and a breathing circuit that organizes gas flow in relation to a subject, the breathing circuit including an inspiratory limb for directing a gas stream towards the subject and optionally an expiratory limb for receiving exhaled gas from the subject, the system comprising:
a. anesthetic delivery means (for example an anesthetic vaporizer or liquid injector) for introducing an adjustable amount of anesthetic (optionally vaporized anesthetic or a fine liquid stream) into the breathing circuit, the breathing circuit adapted to receive the anesthetic via an inlet port in the breathing circuit;
b. a measurement system operatively connected to the breathing circuit including an anesthetic analyzer for analyzing at least the concentration of anesthetic in the gas stream flowing towards the subject and a flow sensor for measuring the rate of flow of the gas stream to the subject; and
c. a control system for receiving input from the measurement system and controlling the amount of anesthetic entering the inlet port including input means for inputting a setting that corresponds to a selected amount of anesthetic in the gas stream flowing to the subject (optionally expressed as a concentration preferably a percentage concentration), the control system utilizing a control algorithm for controlling the amount of anesthetic entering the inlet port based on said selected amount of anesthetic, a flow rate of the gas stream flowing to the subject measured by the measurement system and, where applicable (i.e. where the subject is rebreathing exhaled gas as in e.g. a circle circuit), the concentration of anesthetic already in the gas stream as measured by the measurement system.

Optionally, the breathing circuit is a re-breathing circuit (meaning a circuit organized to enable a subject to re-breath anesthetic-containing exhaled gas; typically a circle circuit that includes a carbon dioxide scrubber), and an expiratory limb for receiving gas exhaled by the subject, the gas stream flowing to the subject including exhaled gas containing anesthetic gas exhaled by the subject. The input means is optionally adapted to set the selected amount of anesthetic in terms of a selectable concentration of anesthetic in the gas stream, and wherein the control system is programmed to top up, if required, the amount of anesthetic already in the gas stream, based on the measured rate of flow of the gas stream, to attain the selected concentration of anesthetic in the gas stream flowing to the subject.

Optionally the control algorithm determines how much anesthetic needs to be added a selected time interval [t]. Optionally, time interval [t] may correspond to each breath. The control algorithm may output a measured volume of gas inspired in each of a series of respective breaths [i] using input from the measurement system and use the volume of each such respective breath [i] and it's pre-top up anesthetic content to compute the amount of anesthetic targeted to enter the inlet port to attain the selected concentration of anesthetic in the gas stream flowing to the subject.

The control system may be programmed to send a control signal to deliver the amount of anesthetic corresponding to the volume and pre-top-up anesthetic content of a respective breath [i] in a subsequent breath [i]+1, any requisite addition of anesthetic to attain or maintain the selected concentration of anesthetic in the gas stream delivered to the anesthetic inlet port, in increments computed on a breath by breath basis, one breath behind.

The anesthetic delivery system includes an anesthetic delivery means which may be a vaporizer which may comprise:
a reservoir containing liquid anesthetic;
a vaporization chamber, to convert the liquid anesthetic into gaseous anesthetic;
a liquid pump, to transfer liquid anesthetic from the reservoir to the vaporization chamber
a gas pump, to transfer the gaseous anesthetic from the vaporization chamber to the anesthetic inlet port.

Optionally, the liquid pump is adapted to transfer anesthetic to the vaporization chamber at an adjustable flow rate controlled by the control system.

Optionally, the flow sensor and gas sensor are located substantially adjacent to one another in the inspiratory limb of the breathing circuit such that substantially all of the gas passing through one sensor passes through the other sensor.

Optionally, the flow sensor is located in the inspiratory limb of the breathing circuit. Optionally, the anesthetic sensor is positioned substantially adjacent to the airway of the patient for measuring the concentration of anesthetic in the gas inspired by a subject as well as the concentration of anesthetic in end tidal exhaled gas.

The system may comprise a carbon dioxide scrubber located upstream of the flow sensor.

Optionally, the anesthetic gas output of the vaporizer to the anesthetic gas inlet is controlled by adjusting the rate of flow of the liquid pump to the vaporizer.

### BRIEF DESCRIPTION OF THE DRAWINGS

The present invention will be further understood and appreciated from the following detailed description taken in conjunction with the drawings in which:

FIG. 1 is a chart illustrating the typical relationship of FmA with respect to time when the anesthetic level is increased in anesthetic breathing systems of the prior art;

FIG. 2 is a schematic illustration of an anesthetic breathing system consistent with an embodiment of the present invention;

FIG. 3 is a schematic illustration of an anesthetic breathing system consistent with another embodiment of the present invention;

FIG. 4 is a flow chart illustrating a method of delivering anesthetic to a patient, consistent with an embodiment of the present invention;

FIG. 5 is a schematic illustration of an anesthetic breathing system consistent with another embodiment of the present invention;

FIG. 6 is a chart showing an example of the timing of algorithm calculation and pump delivery pipelining for topping up gas concentration at fixed intervals of time;

FIG. 7 is a schematic illustration of a portion of the anesthetic breathing system of FIGS. 2, 3, and 5, showing the internal gas flow in the mixer; and

FIG. 8 is a schematic illustration of a medical patient connected to art anesthetic breathing system consistent with an embodiment of the present invention.

### DETAILED DESCRIPTION

Reference will now be made in detail to embodiment(s) of the present invention, examples of which are illustrated in the accompanying drawings, wherein like reference numerals refer to the like elements throughout. The embodiment(s) is/are described below to explain the present invention by referring to the Figures.

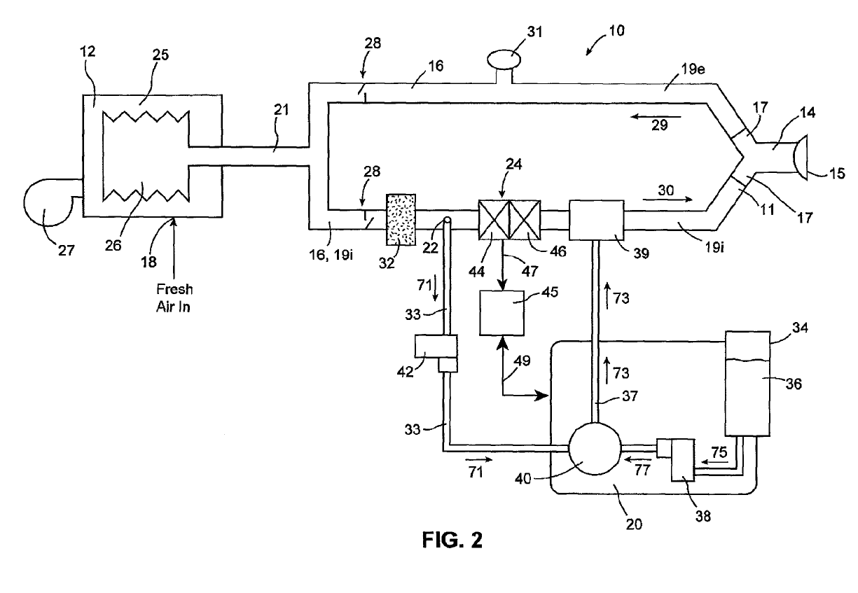

Referring now to FIG. 2, there is shown a schematic view of an anesthetic delivery system 10 consistent with an embodiment of the present invention. The system 10 is used in surgery and other medical procedures to maintain a partially closed breathing environment for the patient, through which a controlled and medically appropriate amount of anesthetic, in the form of a gas, may be administered.

As indicated in the figure, system 10 includes a ventilator 12, optionally a patient airway interface which may be in the form of an endotracheal tube (not shown) or a mouthpiece 15, and a breathing circuit or air conduit 16 that connects and provides a closed path for air flow between the ventilator 12 and a Y-piece 14. Ventilator 12 contains a fresh air input port 18 through which fresh air enters system 10.

The ventilator 12 is a device or machine that minimally maintains a flow of air to the patient. In addition to electrically operated devices or machines, a ventilator may take the form of a manually operated device such as an Ambu bag. In a circle system (explained below), the ventilator 12 typically serves to re-circulate air exhaled by the patient and accordingly delivers a mix of fresh air, exhaled air, and anesthetic back to the patient for inhalation during the inspiratory part of the breathing cycle.

Anesthetic is supplied by a vaporizer 20, which converts or vaporizes the anesthetic from a liquid to a gas, and delivers the gas to circulation in air conduit 16 in system 10. The term “breathing circuit” means a system of one or more gas conduits and valves that is used to direct the flow of gas to the subject on the ventilator. A circle breathing circuit or system typically includes an inspiratory limb including a one way inspiratory valve and an expiratory valve and a common interconnecting limb that together with a Y-piece forms a circle.

System 10 of the present invention also includes a measurement system 24 (“MS”), located in the breathing circuit 16, more conveniently in an inspiratory limb 19i, in the path of the air flow. As will be discussed in greater detail below, measurement system 24 may comprise at least one sensor that detects the percentage concentration of anesthetic in the breathing circuit, conveniently located upstream from the point of entry of gas from vaporizer 20 into air conduit 16. This information may be used by system 10 to adjust the output of vaporizer 20.

According to at least one embodiment of the invention, ventilator 12 contains an enclosure or chamber 25, a bellows 26, and a blower 27. Bellows 26 fits inside chamber 25 and has an open connection with breathing circuit 16 and fresh air inlet port 18. Bellows 26 is sized to hold enough gas to meet the peak inspiratory flow requirements of the patient.

As a result of this configuration, when the patient exhales the exhaled air passes through breathing circuit 16 and may be received in bellows 26. Expansion of bellows 26 draws in fresh air from the atmosphere through fresh gas in line 18. Blower 27 applies pressure against bellows 26, compressing the gas and forcing or pushing it into breathing circuit 16 for inhalation by the patient.

It is to be appreciated that any device that functions to maintain or exchange air flow with a subject may be used as ventilator 12 in the present invention. For example, instead of a ventilator and blower arrangement, a source of pressurized driving gas may be used to drive the contents of a compressible gas reservoir.

As indicated in FIG. 2, Y-piece 14 may be connected to mouthpiece 15 or to an endotracheal tube. The mouthpiece 15 typically covers and forms an airtight seal over the patients airway, i.e. mouth and nose. The base of the Y-piece joins the arms of the which are a pair of diverging or bifurcated tubes 17 that connect to tubes 19 of breathing circuit 16. As shown, dual tubes 19 of air conduit 16 extend a certain length and then re-converge to form a single tube 21 that is fluidly connected to ventilator bag 26 thus forming a circle. System 10 in the embodiment shown is often called a “circle system” to reflect the closed loop configuration of the tubes forming breathing circuit 16.

Each conduit 19 of breathing circuit 16 typically contains a valve 28 that permits air flow in one direction and blocks air flow in the opposite direction. The two one-way valves 28 are oriented opposite from one another, so that air flows in opposite directions in each tube. More particularly, in the example shown in the figures, valve 28 in the top tube 19 permits air flow in the direction from mouthpiece 15 to ventilator 12, as shown by arrow 29. Similarly, valve 28 in the lower branch 19 permits air flow in the direction from ventilator 12 to mouthpiece 15, as shown by arrow 30. Accordingly, only exhaled air flows in upper tube 19, which may be designated as expiratory limb 19e, and only inhaled air flows in lower tube 19, as noted is designated as inspiratory limb 19i. Expiratory limb 19e further includes a mushroom or poppet valve 31, which activates or opens when the total volume of gas in breathing circuit 16 exceeds a predetermined amount or level.

Since exhaled air contains carbon dioxide (CO₂) and is returned to the subject for inhalation, system 10 also includes a carbon dioxide scrubber 32 in inspiratory limb 19i, to scrub out or remove the carbon dioxide from the inhaled gas.

According to some embodiments of the invention, vaporizer 20 comprises a reservoir 34 that holds liquid anesthetic 36, a liquid pump 38, a vaporization chamber 40, and a gas delivery pump 42. Optionally, as shown in the figures and as described further below, gas delivery pump 42 may be placed in a separate gas flow circuit physically outside of vaporizer 20. As described below, a conduit 33 acts as “bias flow” in that it channels gas containing some vaporized anesthetic via a port 22 through the vaporization chamber to maintain gas flow through the vapor flow path to a degree that prevents re-condensation to the liquid phase.

As indicated by arrows 75 and 77 in FIG. 2, liquid anesthetic 36 is transported by liquid pump 38 from reservoir 34 into vaporization chamber 40. The chamber 40 is an enclosure that is heated by a heat source such as an electric element (not shown). The heat converts liquid anesthetic 36 into a gas, which is then transported by gas delivery pump 42 out of vaporization chamber 40 and into air conduit 16. Optionally, as discussed further below, to facilitate the transfer of anesthetic gas to gas pump 42, a carrier gas may be passed through the vaporization chamber. Vaporizer 20 may include an electronic processor such as a microprocessor to control the operation of these components, or alternatively the vaporizer components may be controlled by an external processor as discussed in more detail below.

Anesthetic liquid 36 may be any anesthetic used in surgical procedures. Each type of anesthetic can also be characterized by a parameter constant IC, which is the ratio of the volume of gas produced by vaporization to a given volume of the liquid. Liquid pump 38 may be any pump suitable for transporting liquids from one chamber to another. Preferably pump 38 is a microfluidic type, capable of transporting very small or minute amounts of fluid, on a scale as small 1 ml, for example. Liquid pump 38 is adjustable so that the rate of flow of liquid 36 can be increased or decreased as desired. The adjustments may be made by manual controls such as a knob, and/or by electronic signals such as those issued by a controlling microprocessor in vaporizer 20 or elsewhere in system 10. In particular, increasing the rate of flow of liquid pump 38 will result in more liquid anesthetic 36 being delivered to vaporization chamber 40, and more anesthetic gas delivered to air conduit 16.

Gas delivery pump 42 may be any industrial or medical quality pump capable of transporting gas in microfluidic amounts.

It is to be appreciated that, unlike other systems in which anesthetic gas is delivered to the fresh air inlet at the ventilator, the anesthetic delivery system 10 of the present invention delivers anesthetic gas directly into breathing circuit 16. More particularly, the gas output of vaporizer 20 may be fluidly connected directly to one of the tubes 19i or 19e. Optionally, as shown in the figures and discussed in greater detail below, the gas output of vaporizer 20 travels through air conduit 37 in the direction of arrow 73, and enters air conduit 16 through another element. More conveniently, the vaporizer gas output enters inspiratory limb 19i rather than expiratory limb 19e, as some of the gas in expiratory limb 19e may leave the breathing circuit before reaching the patient.

It is also to be appreciated that, unlike the systems in which anesthetic gas is mixed with fresh air prior to delivery to the breathing circuit, anesthetic delivery system 10 of the present invention delivers anesthetic gas undiluted by fresh air.

Measurement system (MS) 24 includes a flow sensor or meter 44 and a gas sensor 46. As noted, MS 24 is positioned in the air flow path of breathing circuit 16. In this way, the circulating air passes through the sensors, enabling the sensors to perform their measurements conveniently.

Data obtained from flow sensor 44 can be used to determine the size of each breath expressed as a volume, and the breath period (T_{B}), or length of each breath in seconds, as well as the integrated flow in any time period. It may be noted that the reciprocal of the breath period is the breath frequency. A pressure transducer may also be used to determine the beginning and end of any portion of the breathing cycle. For example, if the breath period is six seconds, the breath frequency is 10 breaths per minute. Accordingly, flow sensor 44 may be used to measure either breath period or breath frequency. The related inverse parameter may be calculated as needed by flow sensor 44 itself or by a processor in system 10. Gas sensor 46 measures the concentration of anesthetic gas in the overall circulating gas comprising exhaled air, fresh air, and anesthetic gas, for a given volume of circulating gas. For example, if the reading of gas sensor 46 is 1%, then in 1 ml volume of circulating gas there will be approximately 0.01 ml of anesthetic gas.

The component sensors of MS 24 may be placed in either expiration tube 19e or inspiration tube 19i, but are preferably placed in inspiration tube 19i.

According to some embodiments of the invention, as shown in FIG. 2 the component sensors of MS 24 may be placed adjacent to one another and to the entry point of gas from vaporizer 20. This configuration is convenient to implement since it enables flow sensor 44 and gas sensor 46 to be organized as a single unit. This configuration also facilitates calculation of vaporizer adjustments, as discussed further below. For convenient reference, this embodiment may be referred to as the first embodiment.

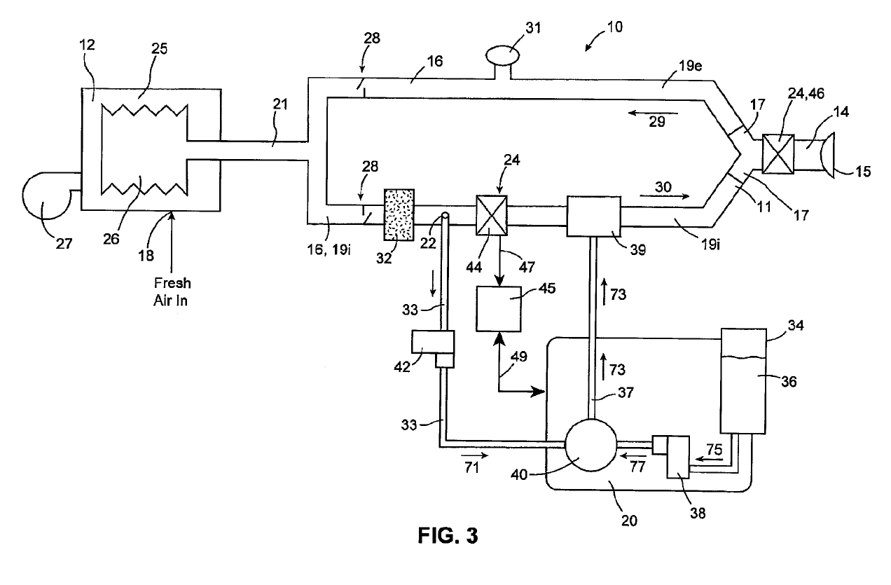

According to some embodiments of the invention, the sensors may alternatively be separated so that they are not immediately adjacent to one another. As shown in FIG. 3, which may conveniently be referred to as the second embodiment, flow sensor 44 may remain in the same position as before, in inspiration tube 19i close to or adjacent to the entry point of gas from vaporizer 20. Gas sensor 46 may be moved to a location close to the patient's mouth for detecting anesthetic in the subject's exhaled end tidal gas. Alternatively, it may be preferable to have a separate sensor for this purpose. This second embodiment is more complex to implement requiring factoring out of the added anesthetic and is also less convenient since the sensors cannot be packaged together.

The parameter FmA, which as noted is the percentage concentration by volume of anesthetic in the air inspired by the patient is optionally upstream from the entry point of gas from vaporizer 20. Alternatively, the anesthetic concentration sensor may be placed in a position optionally close to the patient's mouth or after a patient's Y-piece so that measurement of the end tidal as well as inspired anesthetic concentration is also possible.

The embodiments shown in FIGS. 2 and 3 further include a controller 45. As indicated, controller 45 communicates with measurement system 24 over communication line 47 and with vaporizer 20 over communication line 49. More particularly, controller 45 receives sensor information from sensors 44 and 46, and engages in two-way communication with vaporizer 20 to control the topping up process described in further detail below. Controller 45 is a computer processor such as a microprocessor that may be programmed to execute operational procedures and functions as described further below.

Conduits 33 and 37 provide pathways for gas flow between inspiration limb 19i and vaporizer 20. As noted, gas delivery pump 42 may optionally be placed in either conduit 33 or 37, and is shown in conduit 33. The figures also show a mixer or homogenizer 39, located in inspiration limb 19i upstream from measurement system 24. These elements, and in particular conduit 33 and mixer 39, provide an optional “bias flow” to the anesthetic gas being generated in vaporizer 20, which enhances the operation of system 10.

In the bias flow, a portion of the gas flowing in inspiration limb 19i, as regulated by gas pump 42, branches off or is diverted from inspiration limb 19i and passes through conduit 33 into vaporizer 20. This bias gas is shown by arrow 71 in the figures. Inside vaporizer 20 the diverted gas enters vaporization chamber 40 where it is heated along with liquid anesthetic 36 pumped in from reservoir 34. Accordingly, inside the chamber liquid anesthetic 36 is heated and evaporated, and the evaporated gas is mixed with the bias flow gas. By introducing the lower anesthetic concentration bias flow that is constantly flowing through vaporization chamber 40, the gas concentration in vaporization chamber 40 is lowered or diluted. This dilution helps the evaporation process by reducing the heat and energy required for evaporation, and enabling quicker delivery of anesthetic gas to breathing circuit 16.

More particularly, the bias flow is constantly bringing in new gas and washing away the high concentration of evaporated anesthetic, which allows more anesthetic to be vapourized. The air flow in inspiration limb 19i is typically sporadic, since there is flow during inspiration but not during the expiration phase. Accordingly, in the ordinary course it is possible that a very high concentration of gaseous anesthetic would build up in inspiration limb 19i, making it difficult to vaporize more liquid anesthetic. The effect of bias flow and using bias flow pump 42 is that it enables system 10 to control the dilution more effectively than relying on the pattern of inspiration and expiration as set by the attending physician.

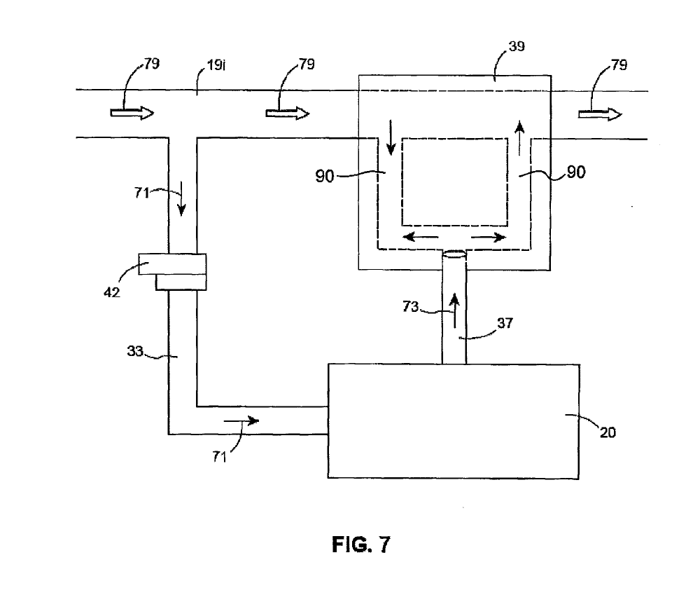

The output anesthetic gas from vaporizer 20 and shown as arrow 73 is then combined, in mixer 39, with the non-diverted gas flowing in inspiration limb 19i. As noted, the flow of gas in inspiration limb 19i is intermittent as there is flow during inspiration and no flow during expiration. This is in contrast to the bias flow of gas diverted through the vaporizer, which is constant. It is to be appreciated that if the two flow paths were just merged there would be uneven concentration—high between two inhalations, and low during the inhalation. In order to homogenize the concentration, mixer 39 holds or stores the anesthetic enriched bias flow until the next inhalation. Then, upon the subsequent inhalation, the gas from both streams are mixed together, thereby reducing the high peaks of concentration and providing a more smooth and level output. FIG. 7 shows the bias flow in more detail, including particularly the circulation of bias flow gas in mixer 39. As seen in FIG. 7, non-diverted gas is represented by arrows 79. A portion of this gas goes through an alternate path 90 which receives vaporized anesthetic from the vaporizer. During expiration, anesthetic vapor, if continuously generated, or to the extent of any residual amounts still leaving the vaporizer, may flow in both directions through the alternate path 90 after entering the mixer via conduit 37, whereas during inspiration flow through the alternate path 90 is unidirectional since a portion of gas flow depicted by arrows 79 is diverted through the alternate path in one direction (the same direction).

It may be noted that for safety purposes the breathing circuits will usually also have a sensor at the patient's mouth, sometimes called a “phase-in verification sensor” (not shown), that measures the true FmA at the point of entry into the patient. There is also usually present at the same point a microbiological membrane filter, sometimes called an “HME filter” (also not shown), to catch undesirable microorganisms to protect the equipment and the patient from contamination.

The operation of anesthetic breathing system 10, according to the preferred embodiment of the invention, will now be described.

At the beginning of the surgical procedure, the anesthetist will select an initial anesthetic concentration level, or FmA, for the patient to receive. The doctor will activate vaporizer 20, and the anesthetic gas concentration in breathing circuit 16 will rise to the selected initial value.

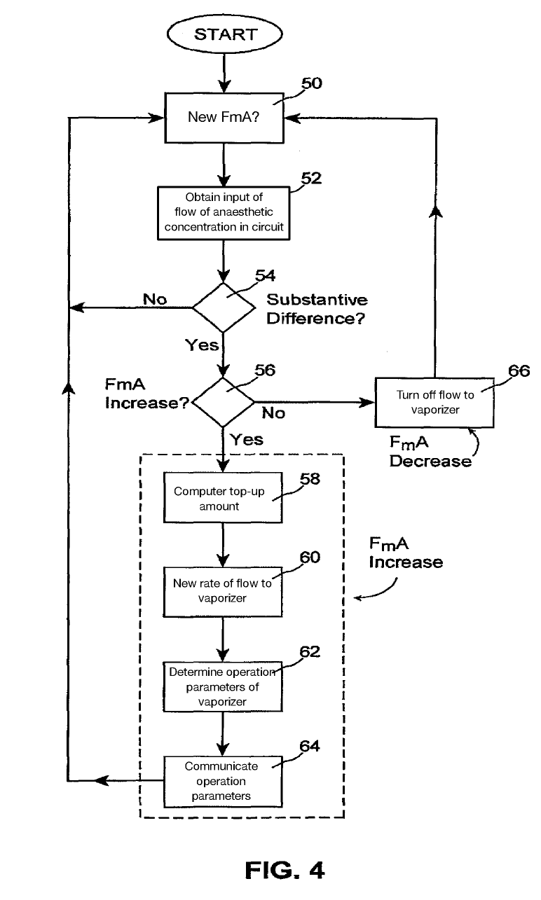

As the surgery proceeds, optionally in every breath taken by the patient, or in any other regular time period, system 10 will evaluate the anesthetic gas concentration in air conduit 16, and take action as required. A flow chart illustrating this method, according to some embodiments of the invention, is shown in FIG. 4.

Beginning at module 50, system 10 checks the selected, desired, or target value of FmA for any change that may have been made by the physician. Next, at module 52, the sensors in MS 24 are used to determine parameters of the circuit gas flow noted above: the size of each breath (typically in ml), the breath frequency (or its inverse, breath period), and the concentration of anesthetic gas passing through the sensor. Subsequently, at decision module 54 system 10 compares the target FmA to the measured concentration of anesthetic gas, and queries whether there is any substantive difference. If the answer is “no”, i.e. the measured concentration is already at the target FmA level, control returns to module 50 and the above sequence is repeated.

As a preliminary matter, it may be noted that when a circle type anesthesia breathing system is operating in the steady state, i.e. when FmA is substantially equal to the selected or target value, the anesthetic concentration of gas flowing through MS 24 will be very close to FmA, i.e. the actual inspired concentration at a reference point 11. This is because the patient absorbs only a small amount of anesthetic in each breath. Further, the primary factor that acts to dilute the anesthetic concentration in the system is the rate of Fresh Gas flow (FGF) into the system, but typically in a circle system FGF is set very low. Accordingly, in the steady state very little anesthetic needs to be added by the system to maintain FmA at the target level, and vaporizer 20 may be operated at a substantially slower rate (after a quasi-steady state is reached) for extended periods of time.

At some point during the surgical procedure the physician may decide that the patient needs more anesthetic, and will proceed to set a new, higher FmA target. As a result, the answer to the query of decision module 54 will be that there is a substantive difference between the new target value (obtained from module 50) and the measured concentration (module 52). Decision module 56 will then determine that the change is an increase in FmA, and passes control to modules 58-64.

Module 58 determines how much anesthetic gas needs to be added to the circulating gas at each breath so that FmA will reach the target value. Module 60 then determines the new flow rate of liquid anesthetic vaporizer 20 by dividing the amount of anesthetic that must be added to each breath by the time taken to deliver a breath. Optionally, in a module 62 system 10 determines the specific operating parameters of one or more elements of vaporizer 20 that have to be changed, if any, to obtain the new flow rate determined in module 60, which may simply be turning on a heating means. Lastly, in module 64 system 10 communicates any changes or new operating parameters to vaporizer 20. Upon completion of this step, control returns to module 50 to repeat the sequence.

In performing the above method of increasing FmA, system 10 of the present invention is “topping up” the amount of anesthetic gas that is known to be circulating in the system. Instead of adding anesthetic through the fresh air flow into the ventilator, which adds a large volume of unneeded air into the system that will be shortly ejected, along with the added anesthetic, the present invention directly tops up the anesthetic concentration that is recovered with an appropriate amount of undiluted anesthetic gas.

This uses substantially less anesthetic, because anesthetic is not being released via mushroom valve 31 as excess gas from the system. This valve is typically open only during exhalation to allow excess gas in the system to leave. Another benefit is that responses to increases in FmA are relatively fast compared to a standard vaporizer. The faster speed results from the fact that when FmA is increased, regardless of the new FmA level, each breath returning to the MS is still topped up by system 10 with the correct amount of vapor to achieve the new FmA almost immediately. A response may be achieved within one to three breaths.

It may be noted that air flow corresponding to a breath arriving at MS 24 could be measured instantaneously, and anesthetic delivered to that breath in accordance with this measurement. In practice, it may be preferable to assess the need for and make anesthetic concentration adjustments in rapid time intervals rather than on a breath by breath basis, for example, time intervals of 0.2 second. However, in practical terms the former procedure would require extremely high pump and vaporization flows, followed by periods during exhalation in which there is little or no anesthetic flow. Therefore, according to some embodiments of the invention, MS 24 measures the entire breath and the control sets to set liquid pump 38 to run continuously at the rate dictated by the amount of anesthetic adjustment needed for the entire breath, even though this means that the pump setting is one breath behind the measurement. As a result, according to some embodiments of the invention, the topping up performed by system 10 is based on sensor readings of the previous breath, and will therefore typically be one breath behind.

An example of the application of the method of the present invention, according to some embodiments, may now be demonstrated. In the example the initial or steady state FmA is 0.6% isoflurane, and the physician decides to raise FmA to a new target of 1% isoflurane.

Beginning at module 50, system 10 reads the selected value of 1% FmA. At module 52, MS 24 obtains three sensor readings:
(1) breath size=500 mL (detected by flow sensor 44),
(2) breath length or period T_{B}=6 seconds, i.e. breath frequency=10 breaths/minute (detected by flow sensor 44), and
(3) concentration of gas entering MS 24=0.6% (detected by gas sensor 46)

The gas concentration of 0.6% is as expected since it is the steady state value.

At decision module 64, the new target FmA of 1% is compared to the measured concentration of 0.6%. The difference of 0.4% is substantive, so control passes to decision module 56, which evaluates whether the selected change in FmA is an increase or decrease. In this case it is an increase, and control continues with modules 58-64.

In module 58 system 10 calculates that the amount of anesthetic gas entering MS 24 each breath is: 500 ml×0.6%, or 500 ml×0.006=3 ml. In order for the gas going to the patient to have a concentration of 1%, the amount of isoflurane gas going to the patient per breath must be 1%×500 ml=5 ml. Accordingly, the amount of gas that must be added for each breath so that FmA will be at the target value is: 5 ml−3 ml=2 ml.

In module 60 the new flow rate of gas output from vaporizer 20 is calculated as 2 ml of gas to be added each breath divided by the 6 second time of each breath. The rate of 2 ml/6 seconds may equivalently be expressed as a flow 20 ml/minute of anesthetic gas into the circuit, on average.

In module 62 system 10 converts the overall vaporizer output flow rate of 20 ml/minute into a flow rate for liquid pump 38. Module 64 communicates this figure to vaporizer 20, and control returns to the beginning of the cycle at module 50.

The algorithm employed by system 10 in module 62 to determine the rate of liquid pump 38 may be described in more detail.

The following terms may be defined:
viaL=rate of liquid anesthetic injection (mL/min), i.e. by liquid pump 38;
viaG=rate of gas anesthetic injection (mL/min), i.e. by gas pump 42 at port 23;
Vt=Tidal Volume (mL) delivered by the ventilator;
Tb=Breath Period (s);
FmA=Actual concentration of incoming anesthetic at the mouth (%);
FcA=Concentration of incoming anesthetic to MS 24 (%);
FwA=Desired inspired concentration of anesthetic in the inspiratory gas (%);
K=Gas volume to liquid volume ratio of anesthetic (constant for a particular agent); and
TU=top up amount per breath (ml) to bring the incoming concentration of the breath up to FmA.

The algorithms in module 62 operate on a breath-by-breath basis, so the above parameters are for a given breath. The algorithms are also different for each embodiment.

The algorithm for the first embodiment (single-case) is as follows. If gas delivery pump 42 were off, the total amount of anesthetic that would be delivered to the patient is: FcA×Vt. However, to achieve an inspired concentration of FwA, the total amount of anesthetic delivered to the patient should be: FwA×Vt. Accordingly, the amount that needs to be topped up during the breath for the breath concentration to reach FwA is:

TU=FwA*Vt−FcA*Vt=(FwA−FcA)*Vt (1)(top-up equation)

To calculate the rate of gas anesthetic to be pumped into the hose in the time of a breath, TU is divided by the breath time Tb, and multiplied by 60 for ml/min:

viaG=(FwA−FcA)*Vt*60/Tb (1a)(gas rate equation)

To convert from gas flow rate to liquid flow rate, viaG is divided by K:

viaL=(FwA−FcA)*Vt*60/(Tb*K) (2)(algorithm rate equation)

Since the pump can only deliver anesthetic to the system, but not remove it, the algorithm for the first embodiment is:

If FwA>FcA,viaL=(FwA−FcA)*Vt*60/(Tb*K) (3)(first embodiment equation)

Else, viaL=0

Preferably, the parameters are measured every breath and the pump rate, viaL, is adjusted every breath.

The algorithm for the second embodiment (dual-case) is as follows. In this situation, the anesthetic delivery is based on the incoming concentration to the mouth. The incoming concentration of the gas at gas delivery port 23 is not known, but it can be calculated based on measurable parameters. The concentration measure at the mouth, FmA, on a given breath, is a result of the pump adding the top-up amount to FcA. Therefore, FcA can be calculated by subtraction, as follows:

FmAn*Vt=FcAn*Vt+viaLn−1*K*Tb/60

FcAn=FmAn−viaLn−1*K*Tb/(60*Vt) (4)(estimation of FcA)

In these equations, viaLn−1 is the rate of liquid anesthetic injection from the previous breath (i.e. breath number “n−1”), and FmAn is the concentration measured by the gas analyzer in the current breath.

On any breath, to bring FcA to FwA, as above in the first embodiment, liquid pump 38 must be set to:

viaLn=(FwA−FcAn)*Vt*60/(Tb*K)

Substituting Equation (4) for FcAn yields:

$$\begin{matrix}viaLn = ( FwA - [ FmAn - viaLn \text{-} 1 * K * Tb \text{/} ( 60 * Vt ) ] ) * Vt * 60 \text{/} ( Tb * K ) \\ = [ FwA - FmAn ] * Vt * 60 \text{/} ( Tb * K ) + viaLn \text{-} 1\end{matrix}$$

Accordingly, the equations (5) for the second embodiment are:

initial value of viaL: viaL0=0

If FwA>FmA: viaLn=(FwA−FmA)*Vt*60/(Tb*K)+viaLn−1

Else: viaLn=0

Returning to the flow chart of FIG. 4, if the target FmA is set to a lower value than the steady state, the outcome of decision module 56 will be a decrease in FmA, and control will pass to module 66. Typically the fastest way to lower anesthetic concentration is simply by turning vaporizer 20 off completely, which may be achieved by turning off liquid pump 38. Accordingly, module 66 sends an electronic signal to vaporizer 20 setting the rate of flow of liquid pump 38 to zero (i.e. viaL=0). Alternatively or in addition, the heating element of vaporization chamber 40 may be turned off. As shown, upon execution module 66 returns control to module 50.

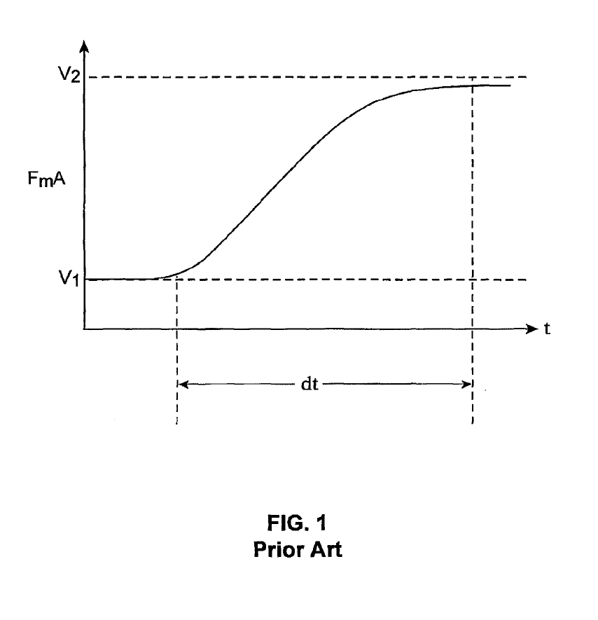

With vaporizer 20 turned off, FmA will decrease exponentially, similar to the manner in which FmA was shown to increase in value in the chart of FIG. 1, but in reverse (i.e. from V₂ to V₁). The time constant of the drop in FmA value would be a function of the rate of flow of fresh air into the system. Over the next few breathing cycles, as FmA drops towards the selected value, system 10 will continue to cycle through steps 50 to 56 since FmA will still be greater than the target concentration. Ultimately FmA will drop below the target, causing execution of FmA increase modules 58-64, which will turn vaporizer 20 back on, in the manner described above.

According to some embodiments of the invention, system 10 may be configured to lower the rate of flow of liquid pump 38, instead of turning it off completely. In that case FmA would decrease towards the target concentration at a slower rate. Alternatively, vaporizer 20 may include a mechanism for actively removing anesthetic from the system, in which case FmA would reach the lower target concentration faster.

As noted, when lowering concentration it is typically advantageous to turn the vaporizer off, so the anesthetic is washed out of the system as fresh air is added. Due to the automatic monitoring of the breathing circuit on every breath, system 10 of the present invention will turn the vaporizer back on automatically when FmA has dropped slightly below the target value.

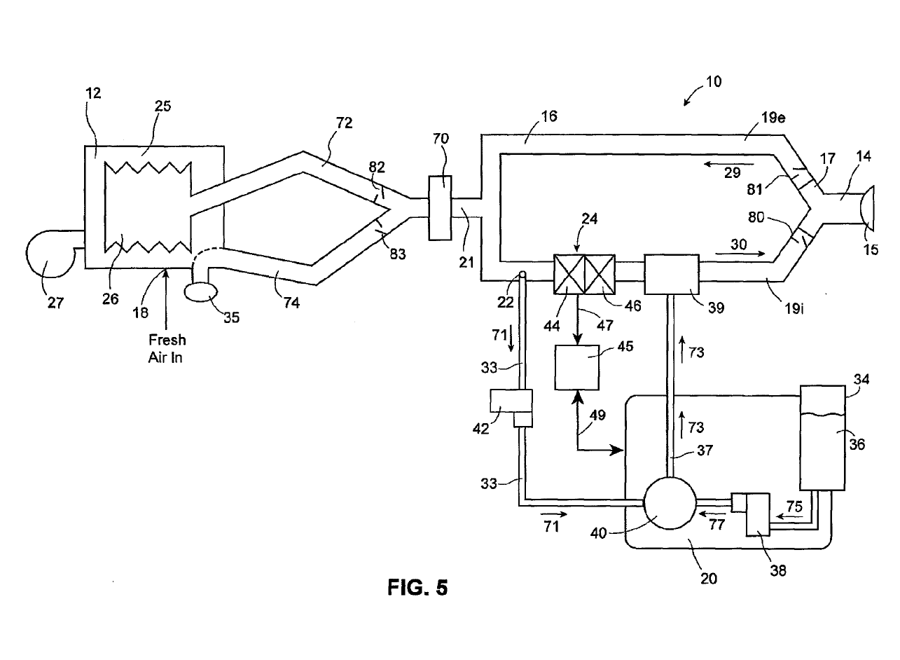

FIG. 5 shows a third embodiment of the present invention, in which anesthetic delivery system 10 is the semi-rebreathing type. As indicated, in this embodiment system 10 includes a reflector or filter 70 in the flow path of single tube 21. Reflector 70 contains a conserving medium or material that has the characteristic of absorbing or trapping anesthetic gas that enters the reflector upon exhalation through expiratory limb 19e and single tube 21, and on subsequent inhalation releasing the trapped anesthetic gas so that it passes back to the patient through single tube 21 and inspiration limb 19i. Reflectors typically have an efficiency rating, usually on the order of about 90-95%, which indicates the percentage of anesthetic that is successfully absorbed and then returned into the system over a period of time.

As indicated in FIG. 5, on the ventilator side of reflector 70 single tube 21 bifurcates into a ventilator tube 72 and a vent tube 74. Ventilator tube 72 provides an air conduit or path into enclosure 25 of ventilator 12. Vent tube 74 provides an air conduit or path to the atmosphere. In FIG. 5 vent tube 74 is shown connected to ventilator 12, as some ventilators contain a port which vents to atmosphere, as shown through a mushroom or poppet valve 35, for this purpose. According to some embodiments, vent tube 74 could alternatively vent directly to atmosphere without passing through ventilator 12.

System 10 further includes a first set of valves 80 and 81 positioned in inspiration limb 19i and expiratory limb 19e, respectively, at approximately the point where limbs 19 meet diverging tube 17. There is further a second set of valves comprising valve 82, in ventilator tube 72, and valve 83, in vent tube 74. The valves are controlled by controller 45 such that upon inhalation valves 80 and 82 are open, allowing air to flow in inspiration limb 19i in the direction of arrow 30, and valves 81 and 83 are closed, blocking air flow. Upon exhalation, valves 81 and 83 are open and valves 80 and 82 are closed, allowing air to flow in expiratory limb 19i in the direction of arrow 29.

In operation, when the patient inhales fresh air flows from ventilator 12 through reflector 70, picking up trapped anesthetic gas. This air then passes through single tube 21 and into inspiration limb 19i. Since valve 81 is closed, air does not flow through expiratory limb 19e. The flowing air passes through measurement system 24, which senses the gas concentration and flow parameters. These figures are read by controller 45, which calculates the appropriate vaporizer output to top up the anesthetic gas already in the system, so that the gas concentration reaching the patient at mouthpiece 15 is substantially the same value as that selected by the attending medical staff. Upon exhalation by the patient, exhaled air flows from mouthpiece 15 through expiratory limb 19e and single tube 21 into reflector 70, which traps most of the anesthetic gas. The exhaled air less the anesthetic gas passes through reflector 70 and vents to atmosphere through vent tube 74 and mushroom valve 35. Accordingly, system 10 of FIG. 5 can be referred to for convenience as a “semi-rebreathing” system because the patient rebreathes only part of the air that he or she exhaled, i.e. the exhaled anesthetic gas, but not the exhaled air itself.

As noted above, the calculation of the amount of anesthetic gas to add may be used, according to some embodiments, to top up the breath on the subsequent cycle rather than the same cycle.

According to some embodiments of the invention, instead of obtaining a new calculation every breath, a new cycle may be commenced at a fixed interval, such as for example 500 ms. After the first 500 ms, the speed of liquid pump 38 is updated and a new calculation cycle is started.

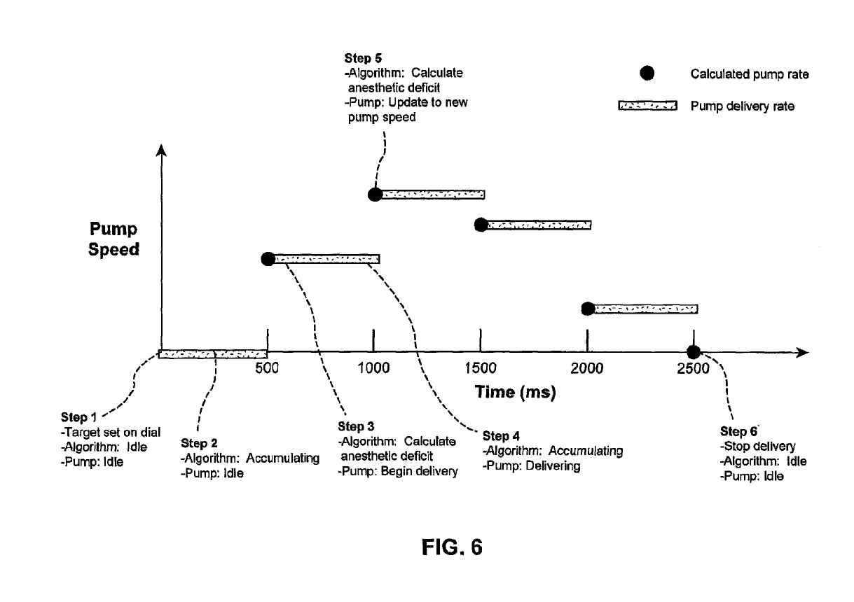

It is useful to consider the interaction of the algorithm and control of liquid pump 38 for this type of calculation. FIG. 6 details the interaction between algorithm calculations and pump speeds during each 500 ms interval. Initially, the instrument dial is parked in Standby. At Step 1, the dial is turned to target a particular concentration. The algorithm then starts accumulating incoming gas data during Step 2. At the end of 500 ms in Step 3, an algorithm calculation is performed to determine the anesthetic deficit from the previous interval. The pump begins to deliver anesthetic to make up the deficit. During Step 4, the pump continues to deliver at a constant rate while the algorithm is once again accumulating data for the next iteration. At the end of another 500 ms interval in Step 5, a new pump speed is calculated and the pump delivery rate is updated. This continues every 500 ms. In Step 6, the dial is turned to Standby and the pump is stopped. In summary, a new pump speed is calculated every 500 ms. During the subsequent 500 ms period, the pump is delivering at a constant rate determined by the result of the latest algorithm calculation. Due to the accumulation phase, the delivery always lags by 500 ms.

An advantage of topping up based on calculation at regular intervals is that it does not depend on breath detection.

As noted, the topping up algorithm calculates the amount of anesthetic to be delivered such that the inspired concentration is equal to the desired level. Since anesthetic delivery system 10 is only capable of adding anesthetic to a system, not removing any, the algorithm is only valid when the incoming concentration is less than the inspired concentration.

In determining the algorithm for the semi-rebreathing embodiment of FIG. 5, a few terms may be defined to aid in the derivation.

Let:

τ=algorithm time interval
FcA=incoming concentration of anesthetic in volume % (most recent interval)
FwA=desired inspired concentration of anesthetic in volume % (set by user)
V=inspired gas volume in last algorithm time interval
A=volume of anesthetic vapour
va=volume of anesthetic liquid
K=anesthetic gas to liquid ratio (A/va)

In the time interval τ, the amount of anesthetic vapour that should be dosed to the patient is determined by V and FwA. The formula for calculating the required anesthetic vapour (AW) is:

A_{W}=V×FwA

For a circle-system, there may already be anesthetic present in the breathing circuit. The total amount of anesthetic vapour already present in the breathing circuit (AC) is:

A_{C}=V×FcA

The amount of anesthetic gas (A) to be topped up is then the difference between the two:

$$\begin{matrix}A = {A}_{W} - {A}_{C} \\ = V \times FwA - V \times FcA \\ = ( FwA - FcA ) \times V\end{matrix}$$

The total amount of anesthetic liquid (va) to be delivered in a breath is related to A by K:

$$\begin{matrix}va = \frac{A}{K} \\ = \frac{( FwA - FwC ) \times V}{K} , KISO = 194.6 , KSEVO = 182.4 .\end{matrix}$$

This algorithm will also work for open systems because FcA will always be equal to zero. Moreover, as long as the dose va is delivered within a reasonable time, the overall amount of anesthetic delivered is correct. This value τ may be variable so that it matches a breath, or it can be a fixed interval. A fixed interval of τ=500 ms is chosen for this particular implementation. The rate of delivery, via, for the subsequent 500 ms interval after acquisition is:

$$\begin{matrix}via = \frac{va}{τ} \\ = \frac{( FwA - FwC ) \cdot V}{K \cdot τ}\end{matrix}$$

### Calculation of V and FcA

The volume of air delivered can be determined by integrating the air flow signal over a breath.

$$V = {\int }_{0}^{Tb} V ( t ) dt ,$$

where V(t) is the instantaneous air flow.

The incoming concentration of anesthetic, FcA may vary in time and cannot be observed directly from a sensor and must be calculated by integrating the concentration over a time interval.

$$FcA = \frac{{\int }_{0}^{Tb} A ( t ) V ( t ) dt}{V}$$

where A(t) is the instantaneous concentration of anesthetic and V(t) is the instantaneous air flow. The numerator is the total volume of anesthetic passing through, while the denominator is the total volume of all gases passing through. In the case of multiples agents in system, the algorithm will calculate dosage based upon the canister identification.

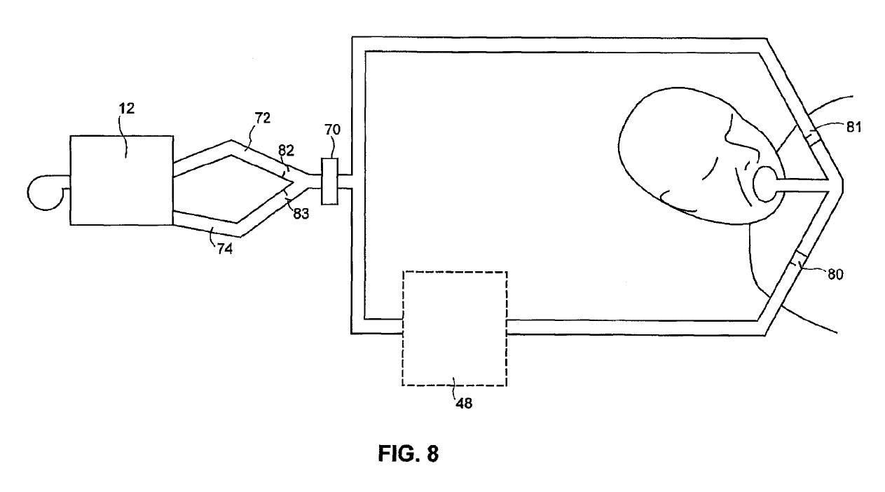

FIG. 8 shows a subject 13 breathing through a third embodiment of anesthetic delivery system 10. A dashed line 48 represents the fact that in practice, vaporizer 20, gas pump 42, controller 45, and the other elements shown may be enclosed in a single housing unit or container 48. As discussed, upon exhalation the exhaled air passes through valve 81 and expiratory limb 19e. Due to this configuration the dead space in front of subject 13 is limited to the space taken up by mouthpiece 15 and y-piece 14. As a result, air conduits 19i and 19e may be made with a relatively greater length, and the various system components, including reflector 70, may be placed in a convenient location. For example, they may be placed behind the patient's bed. More particularly, reflector 70 and vaporizer 20 may be placed away from the subject's mouth, and accordingly do not interfere with any procedures that may need to be performed by the medical staff. It is to be appreciated that in this way, system 10 of the present invention removes the problem of interference from in-line vaporizers and reflectors, while at the same time keeping dead space to a minimum.

The term “anesthetic return system” means a portion of an anesthetic delivery system adapted for receiving exhaled gas and returning at least a portion of the exhaled gas to the subject. Anesthetic is thus returned to the subject by removing a substantial proportion of the anesthetic from the exhaled gas stream and directing it back to the subject, for example by using an anesthetic reflector in the circuit, or returning exhaled gas to the subject, for example using a re-breathing or circle type circuit configuration which preferably includes a carbon dioxide scrubber.

The term “anesthetic reflector” means a device containing a material for releasable sorption of gas-borne anesthetic agent, for example an activated carbon. A typical anesthetic reflector has a housing containing or defining at a least a part of a gas flow channel through a filter comprising a material for releasable sorption of gas-borne anesthetic agent, and two externally accessible ports including a patient-side port and a ventilator-side port. The patient side port may be connected to a patient airway interface via one or more gas conduits of a breathing circuit. The breathing circuit may be a single limb circuit, or as disclosed herein, separate inspiratory and expiratory limbs that are connected to the patient-side port, for example, via a Wye connector. Various embodiments of an anesthetic reflector are well known and referenced herein and in other patent and scientific literature, for example CA 2271385, WO2006/009498, and U.S. Pat. No. 7,077,134 (and patents cited therein) which discloses how a suitable filter material may be interposed within alternate flow paths, for example an inspiratory limb and an expiratory limb.

The term “limb” is used to refer to a gas conducting conduit. The terms inspiratory and expiratory are used to modify the term “limb” to denote a function of a section of conduit in terms of receiving expired gas and delivering inspired gas respectively without necessarily implying that a single conduit cannot perform both functions. It will be appreciated that topping up gas flowing to the subject requires knowledge of the concentration of anesthetic already in this gas flow. According to exemplified embodiments of the invention, the inspiratory and expiratory portions of the breathing include separate portions for receiving expiratory gas and delivering inspiratory gas, for example, for convenience so that a scrubber, where required (if a reflector is used exhaled gas can be vented to atmosphere) can be placed in a separate expiratory limb (e.g. to avoid unnecessary dead space and placing a scrubber at mouth).

The term ‘mixer’ is used interchangeably with ‘homogenizer’ and is used to refer to any portion of a respiratory gas delivery system that is specially adapted to making the concentration of anesthetic in a segment of a gas flow stream more homogenous, thereby for example, reducing high peaks of concentration and providing a more smooth and level output of anesthetic to the patient.

The term “flow sensor” and “flow meter” are used interchangeably and include any device that measures at least one parameter from which a measure of rate of flow can be determined.

Although selected embodiment(s) of the present invention has/have been shown and described, it is to be understood that the present invention is not limited to the described embodiment(s). Instead, it is to be appreciated that changes may be made to this/these embodiment(s) without departing from the principles and spirit of the invention, the scope of which is defined by the claims and the equivalents thereof.

1.-37. (canceled)

38. An anesthetic delivery system (10) for use in conjunction with a ventilator (12), and a breathing circuit (16), the breathing circuit (16) organizing gas flow in relation to a subject and operatively connected to an anesthetic return system (19e, 70) for re-utilizing anesthetic exhaled by the subject and including an inspiratory limb (19i) for directing a gas stream from the ventilator (12) towards the subject, the system comprising:
an anesthetic delivery device (20) operable for introducing a variable amount of anesthetic into the breathing circuit in a vaporized form;
a homogenizer (39) having an anesthetic inlet port and configured for distributing the anesthetic in vaporized form into the gas stream flowing towards the subject, the homogenizer receiving into the anesthetic inlet port the anesthetic in vaporized form directed by the breathing circuit from the anesthetic delivery device, such that anesthetic vapor is received in the inspiratory limb (19i) in a portion of the gas stream passing through the homogenizer, the homogenizer (39) configured to be fluidly connected to the inspiratory limb (19i) of the breathing circuit (16) such that anesthetic vapor that enters the homogenizer (39) during exhalation is stored in the homogenizer (39) during exhalation and is distributed in the gas stream directed towards the subject, via the inspiratory limb (19i), during inhalation;
a measurement system configured to be operatively connected to the breathing circuit for continuously measuring flow and anesthetic content of the gas stream, the measurement system comprising an anesthetic analyzer (46) for analyzing a concentration of anesthetic in the gas stream flowing towards the subject including anesthetic returned via the anesthetic return system, and a flow sensor (44) for determining a rate of flow of the gas stream to the subject;
a control system for receiving input from the measurement system and controlling the amount of anesthetic entering the inlet port from the anesthetic delivery device, the control system operatively associated with an input device for inputting a setting that corresponds to a desired amount of anesthetic in the gas stream reaching the subject, the input device configured for setting the desired amount of anesthetic in terms of a selectable concentration of anesthetic in the gas stream reaching the subject;
the control system (45) operable to execute a control algorithm that controls the amount of anesthetic entering the anesthetic inlet port based on the setting, and based on the rate of flow of the gas stream flowing to the subject and the concentration of anesthetic in the gas stream, as determined by the measurement system, such that anesthetic already in the gas stream flowing to the subject is supplemented to attain a level of anesthetic reaching the subject that corresponds to the setting.

39. An anesthetic delivery system according to claim 38, wherein the breathing circuit includes a carbon dioxide scrubber and an expiratory limb for receiving gas exhaled by the subject, the expiratory limb and the carbon dioxide scrubber fluidly connected to the inspiratory limb such that the gas stream flowing to the subject via the inspiratory limb includes exhaled gas containing anesthetic gas exhaled by the subject.

40. An anesthetic delivery system according to claim 38, wherein the control algorithm:
(a) outputs a value corresponding to a volume of gas inspired in each of a series of respective breaths [i] using input from the measurement system; and
(b) uses the volume of each such respective breath [i] and its pre-top up anesthetic content to compute the amount of anesthetic set to enter the anesthetic inlet port to attain the desired amount of anesthetic in the gas stream reaching the subject.

41. An anesthetic delivery system according to claim 38, wherein the control algorithm:
(a) outputs a value corresponding to a volume of gas inspired measured for each of a series of consecutive time intervals [t] using input from the measurement system; and
(b) uses a respective volume of gas measured for a respective time interval [t] and its pre-top up anesthetic content to compute the amount of anesthetic required to attain the desired amount of anesthetic in the gas stream reaching the subject;
where the control system is programmed to top-up the anesthetic already in the gas stream within a next ensuing time interval [t+1] or a plurality of next ensuing time intervals, as required, to attain the desired amount of anesthetic in the gas stream reaching the subject.

42. An anesthetic delivery system according to claim 40, wherein the control system is programmed to send a control signal to the anesthetic delivery device to signal the anesthetic delivery device to deliver an amount of anesthetic corresponding to difference between the desired amount of anesthetic and the amount of anesthetic already in the gas steam from the anesthetic return system in a respective breath [i], in a subsequent breath [i+1], any requisite addition of anesthetic to attain or maintain the selected concentration of anesthetic in the gas stream delivered to the anesthetic inlet port, added in increments computed on a breath by breath basis, one breath behind.

43. An anesthetic delivery system according to claim 38, wherein the anesthetic delivery device is a vaporizer which comprises:
a reservoir containing liquid anesthetic;
a vaporization chamber, to convert the liquid anesthetic into gaseous anesthetic;
a liquid pump for transferring liquid anesthetic from the reservoir to the vaporization chamber, the liquid pump being adapted to transfer anesthetic to the vaporization chamber at an adjustable flow rate controlled by the control system; and
a gas pump, to transfer the gaseous anesthetic from the vaporization chamber to the anesthetic inlet port.

44. An anesthetic delivery system according to claim 38, wherein the breathing circuit includes a reflector, and a separate expiratory limb for receiving gas exhaled by the subject, the reflector operatively connected to the expiratory limb for reversibly trapping or adsorbing anesthetic in gas exhaled by the subject and between the inspiratory limb and the ventilator, wherein the ventilator drives inspiratory gas through the reflector into the inspiratory limb such that the gas stream flowing to the subject via the inspiratory limb includes anesthetic gas trapped by the reflector.

45. An anesthetic delivery system according to claim 44, wherein the reflector is positioned in the breathing circuit between the inlet port and the ventilator.

46. An anesthetic delivery system according to claim 44, wherein the reflector is positioned in the breathing circuit between the measurement system and the ventilator and wherein the control system is programmed to top-up the anesthetic returned to the inspiratory limb from the reflector.

47. An anesthetic delivery system according to claim 44, wherein the reflector is positioned in the breathing circuit between the homogenizer and the ventilator.

48. An anesthetic delivery system according to claim 38, wherein the control algorithm includes a top-up algorithm for setting the rate of flow of anesthetic liquid from the liquid pump to the vaporizer, the top-up algorithm derived from the mathematical relationship:
(FwA−FcA)*Vt*60/(Tb*K)
wherein:
FwA is the selected concentration of anesthetic in the inspired air;
FcA is the measured concentration of gaseous anesthetic;
Vt is a tidal volume of air delivered by the ventilator;
Tb is the breath period; and
K is a constant comprising a ratio of gas volume to liquid volume for the specific anesthetic used in the method.

49. An anesthetic delivery system according to claim 38, wherein the control algorithm includes a top-up algorithm for setting the rate of flow of anesthetic liquid from the liquid pump to the vaporizer, the top-up algorithm derived from the mathematical relationship:
(FwA−FmA)*Vt*60/(Tb*K)+viaLn−1
wherein:
FwA is a selected concentration of anesthetic in the inspired air;
FmA is an actual concentration of incoming anesthetic at the subject's airway;
Vt is a tidal volume of air delivered by the ventilator;
Tb is a period of the subject's breath;
K is a constant comprising a ratio of gas volume to liquid volume for the specific anesthetic used in the method; and
viaLn−1 is the rate of flow of the liquid pump in the previous breath.

50. An anesthetic delivery system according to claim 38, comprising a pressure transducer for determining the beginning and end of an inspiratory cycle.

51. An anesthetic delivery system according to claim 38, comprising conduits (33, 37) that provide pathways for a gas flow between the inspiration limb (19i) and the anesthetic delivery device (20), wherein the homogenizer (39) and the conduits (33,37) provide a bias flow to the anesthetic vapor being generated in the anesthetic delivery device (20), and wherein the homogenizer (39) stores the bias flow enriched with anesthetic vapor during exhalation until the next inhalation.

52. An anesthetic delivery system according to claim 38, comprising an inspiratory tube (19) of the inspiratory limb (19i), the flow sensor (44) and the anesthetic analyzer (46) connected to the inspiratory tube (19) between the anesthetic inlet port and the ventilator.
